(19) United States
(12) Patent Application Publication
(10) Pub. No.: US 2010/0087805 A1
(43) Pub. Date: Apr. 8, 2010

(54) Dispensing device for pressurized containers for the application of cryogenic coolant

(75) Inventors: Mauro Citterio, Sesto Fiorentino (IT); Luciano Ottanelli, Sesto Fiorentino (IT)

Correspondence Address: Themis Law, 7660 Fay Ave Ste H-535, La Jolla, CA, 92037, US

(73) Assignee: SIXTEM LIFE S.R.L., Sesto Fiorentino (IT)

(21) Appl. No.: 12/524,570

(22) PCT Filed: Jun. 20, 2007
(86) PCT No.: PCT/IT07/00440

§ 371 Date: Oct. 16, 2009

(30) Foreign Application Priority Data
Jan. 31, 2007 (IT) PD2007A000025

(51) Int. Cl. A61B 18/02

(52) U.S. Cl. 606/22

(57) ABSTRACT

The invention relates to a new dispensing device for pressurized containers of coolants for cryotherapy, with a manual opening/closing valve, which includes a dispenser body suited to be permanently fixed to said container, a valve-opening element and a cap with teeth resting against said valve-opening element to cause the opening of the valve. In one embodiment, at least one tube or duct for application of the coolant, at one end of which at least one applicator pad or filter is partially and removably inserted, is permanently built in and integral with said dispenser body.

### FIELD OF THE INVENTION

The present invention concerns medical devices for cryotherapy in the treatment of skin lesions and warts, and in particular concerns a new dispensing device for pressurized containers for the application of cryogenic coolant.

### BACKGROUND OF THE INVENTION

Cryotherapy is a therapeutic process, in which very cold fluids are used for the local treatment of skin lesions like warts or other less or more serious skin diseases.

The controlled and punctual application of coolant to the lesion causes the freezing of the intracellular water and the alteration of proteins and enzymes causing, over the therapeutic application and successive thawing time, the detachment of the epidermis from the dermis without damaging the latter.

The advantages offered by cryotherapy are therefore numerous. First of all, anesthesia of the area to be treated is not usually required and furthermore, in most cases, no scars appear and there is no need for further post-operative medications or treatments.

One of the fluids most used in cryotherapy is liquid nitrogen at the temperature of −196° C., while the use of freon, which is a halogenated gas, has been recently restricted because it is considered a pollutant.

The use of other fluids like carbon dioxide and nitrous oxide is also known, which however require more complex and expensive preparation and conservation processes.

At present, the coolant to be used in cryotherapy is kept in pressurized containers provided with spray dispensers with a manual opening/closing valve.

The known containers also comprise a removable application duct or tube, at whose end there is an applicator pad suited to absorb and convey the coolant that has flowed out of the container.

Said applicator pad soaked with coolant is applied to the area to be treated, which thus freezes, causing the breakage of the cellular membranes and the successive detachment of the epidermis from the dermis.

In the known containers, said duct or tube is removably applied to the dispenser at the moment of use, which means that the user must first of all apply the tube, then position the pad and finally dispense and apply the coolant.

One of the drawbacks of by the known devices lies in that the coolant may be spread out in an uncontrolled manner, for example during application or removal of the tube, thus causing a waste of material and above all a risk of injuries or lesions to the operator or the patient.

Furthermore, it is necessary for the operator to act rapidly when dispensing the coolant, in order to prevent its temperature to increase excessively compared to the optimal values, which would reduce the effectiveness of the treatment.

The use of the known containers, which comprise removable parts like tubes and pads, often makes the operation rather complex and for this reason the operator works less rapidly.

Furthermore, he/she must wait the technical time to make sure that the pad is completely and sufficiently soaked with coolant before proceeding to the application.

### SUMMARY OF THE INVENTION

In order to overcome the drawbacks described above, a new type of dispensing device for pressurized containers has been designed and carried out, which is suitable for the application of a cryogenic cooling gas in the therapeutic treatment of skin lesions.

The main object of the invention is to provide a dispensing device that can be used in total safety for the operator and the patient, since uncontrolled leakages of coolant are prevented by an activator cap or plug, with which the fluid is dispensed in a closed space in order to soak the pad or applicator filter.

Another object of the present invention is to provide a device that can be used in a quick and straightforward manner, since the duct or tube of the coolant to be dispensed is built in and integral with the body of the dispenser, and therefore it is not necessary to install the tube in advance, a procedure which causes an inevitable waste of time and possible uncontrolled fluid leakages.

Good hygienic conditions are guaranteed by the possibility of replacing the pad or filter partially housed in said tube after every application.

A further object of the present invention is to speed up the application procedure, thus reducing to a minimum any decrease in the fluid temperature before application to the skin and thus increasing the effectiveness of cryotherapy to the benefit of the patient.

One of the advantages offered by the present invention, resulting from the use of pads or filters made of a highly absorbing material with a high capacity to convey the coolant, lies in that it is possible to obtain the immediate cooling of the pad.

A further advantage offered by the present invention lies in that the operator can identify quickly and with certainty the moment when the pad or filter is properly soaked with coolant and therefore ready for immediate application. A further advantage of the invention lies in that it is made of materials that have suitable mechanical and chemical resistance to the substances used and are resistant to low operating temperatures.

These and other direct and complementary objects have been achieved through the construction of a new dispensing device for pressurized containers with manual valve for the application of cryogenic cooling gas in cryotherapy, comprising a dispenser body with a built-in and integral tube, in which it is possible to partially insert a removable cylindrical applicator pad or filter, suited to absorb and covey the coolant, wherein a safety activator cap or plug is suited to close the dispenser body, thus preventing accidental fluid leakages, or to open the valve causing the controlled delivery of the fluid that, conveyed through said tube, is immediately absorbed by said pad or filter.

Said activator cap comprises a casing suited to be positioned to close the dispenser body, to which it can be removably connected, and an inner channel, integral with the upper part of the cap, suited to house said tube or duct of the dispenser body.

The cylindrical wall of said inner channel of the cap comprises, on its lower edge, one or more teeth or projections suited to be inserted in corresponding through slots or holes made in said dispenser body.

A downward pressure exerted on said cap—correctly positioned—causes said teeth or projections to be completely or partially inserted in said slots, so that they rest on an element, hereinafter called valve-opening element, positioned between said dispenser body and the upper edge of the container, connected to said valve.

The pressure exerted on said valve-opening element causes said valve to open, which makes the fluid flow out through said tube or duct integral with the dispenser body.

The pad or filter partially inserted in said tube absorbs the fluid and conveys it to the top, getting completely soaked with said fluid.

In order to maximize the capacity of the pad to absorb and convey the coolant, according to the invention said pad or filter is made of cellulose or open-cell foam, preferably polyurethane S518.

The inner channel of the cap, which contains said tube completely, keeps the inserted pad or filter in the correct position, thus preventing it from being expelled due to the gas pressure.

Said cap or plug is made of a plastic material that has suitable mechanical, chemical and heat resistance, and is preferably transparent, so that the user, once having activated the cap and caused the fluid to flow out, can check from the outside if the pad is soaked and therefore ready to be used.

In fact, the outflow of fluid and the absorption of the same by the pad makes the condensate deposit on the inner surface of the cap near the pad itself, thus making it clear that the pad is soaked and ready to be used.

Said cap and/or said dispenser body and/or said container is/are provided with ribs and/or grooves for the correct alignment and positioning of the cap. In particular, according to the invention said cap can be aligned and arranged on said dispenser body in two different positions. In position “OFF”, the cap applied to said dispenser body is rotated so that said teeth do not fit in the corresponding slots made in the dispenser body and therefore the fluid cannot be dispensed.

In position “ON”, the cap is rotated for example by 180° with respect to position “OFF”, so that said teeth are aligned with said slots, and so that by pressing the cap towards the container the user can insert said teeth in said slots and activate said valve-opening element, thus dispensing the fluid.

To guide the user and allow him/her to arrange the cap correctly in the closing or opening position, the two correct positions are indicated on the cap by means of marks, symbols or the like.

### BRIEF DESCRIPTION OF THE DRAWINGS

The characteristics of the new dispensing device will be highlighted in greater detail in the following description, with reference to the drawings attached as non-limiting examples.

FIGS. 1a and 1b show two vertical sections of the dispenser body (A), while FIGS. 1c and 1d are a top view and a bottom view.

FIGS. 2a-2c show a side view, a cross section and a bottom view of the valve-opening element (B) only.

FIG. 3 shows a section of the dispenser body (A) assembled with the valve-opening element (B).

FIG. 4a shows a 3D view of the activator cap (T) only, while FIGS. 4b-4e show a side view, a cross section, a top and a bottom view of the same. FIGS. 5a-5c schematically represent the operation of the activator cap (T), which is in position “OFF” in FIG. 5a and in position “ON” in FIGS. 5b and 5c.

FIGS. 6a and 6b show side views of the cap where it is possible to see the alignment of the reference marks present on the cap (T) and on the dispenser body (A), with the cap in position “OFF” and “ON”, respectively.

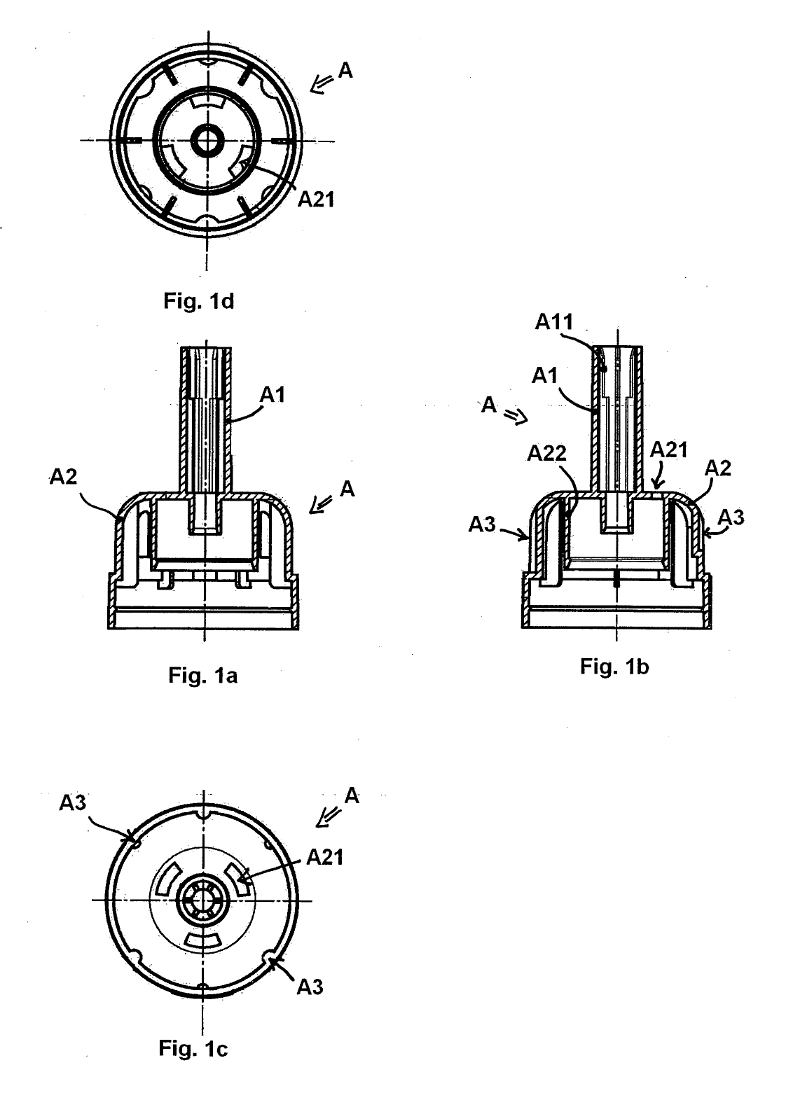

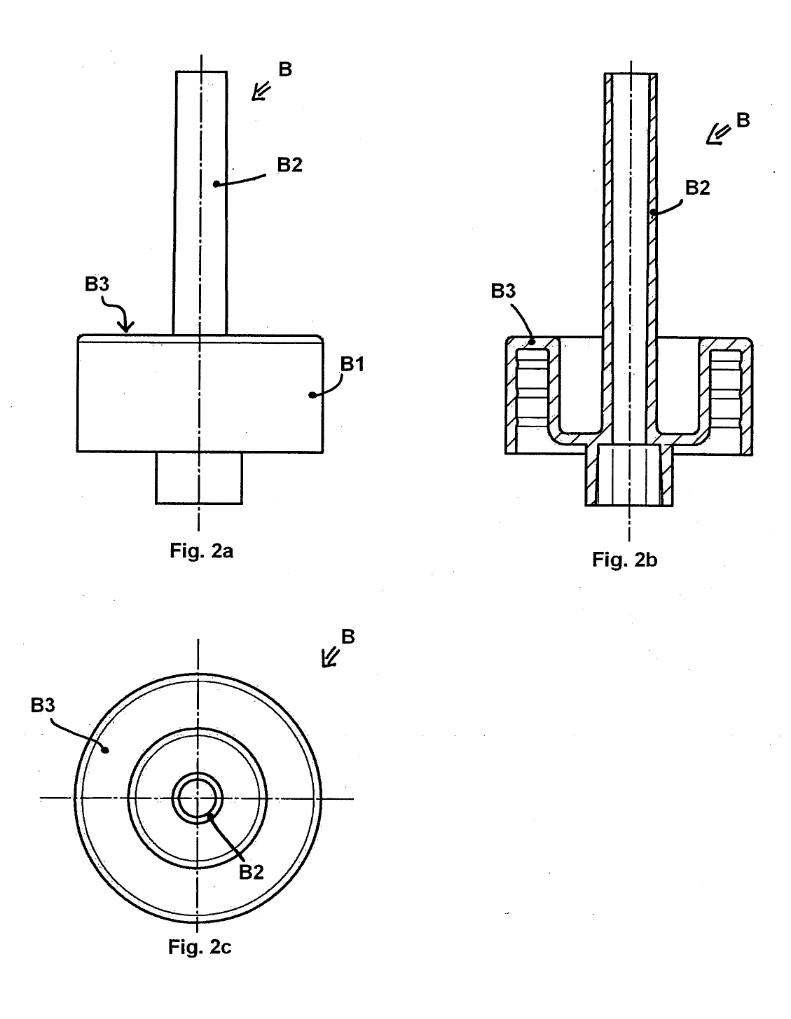

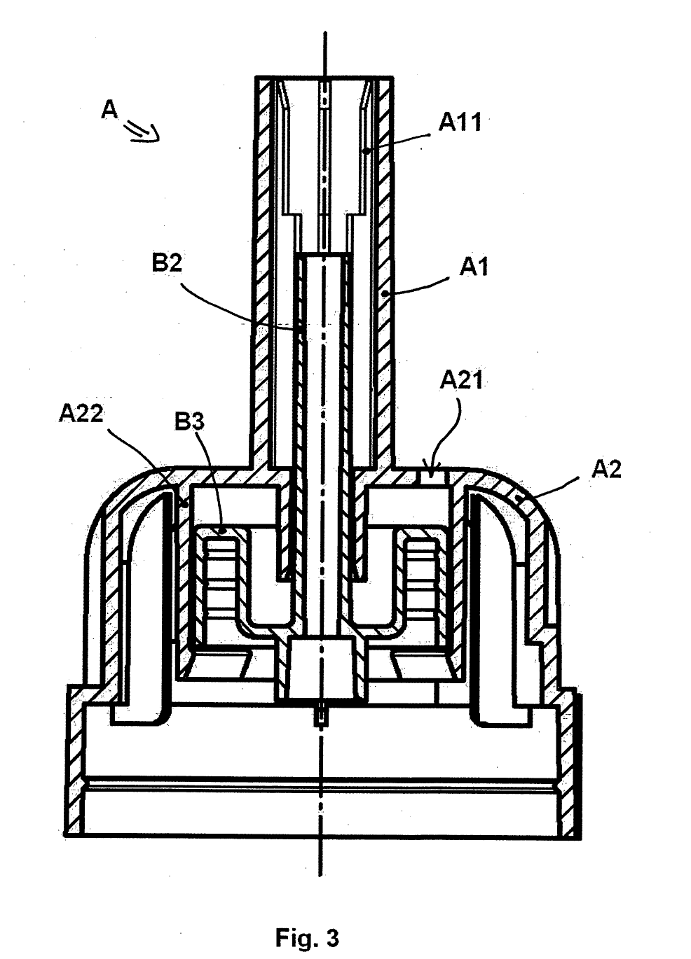

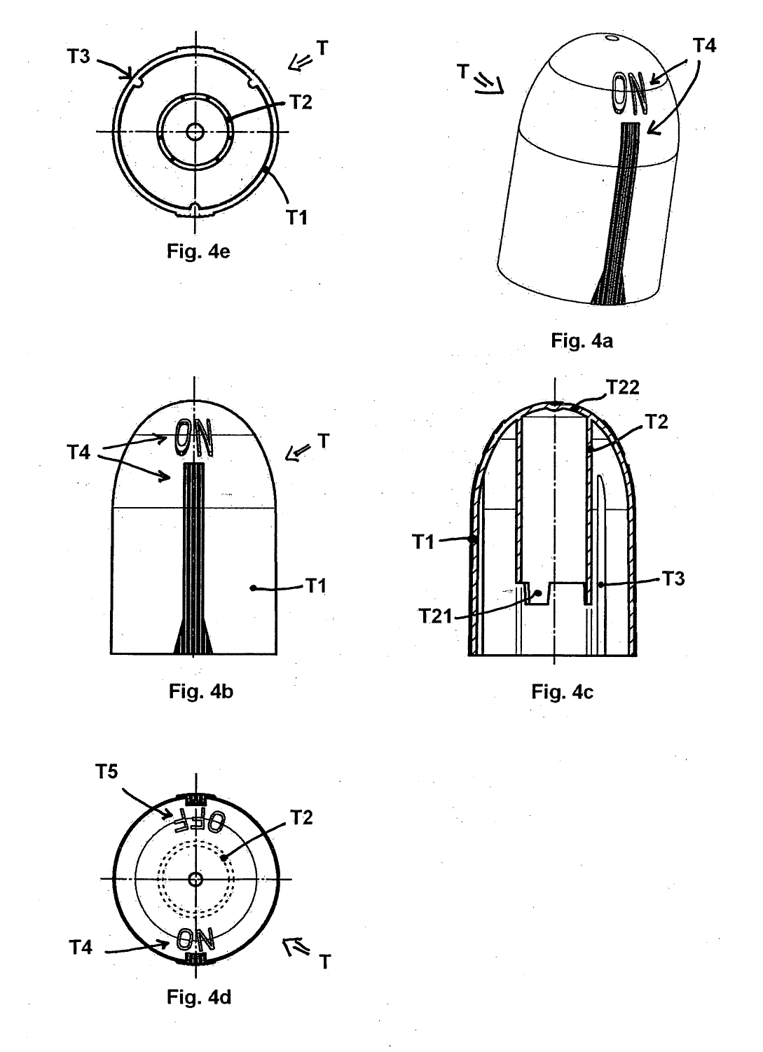

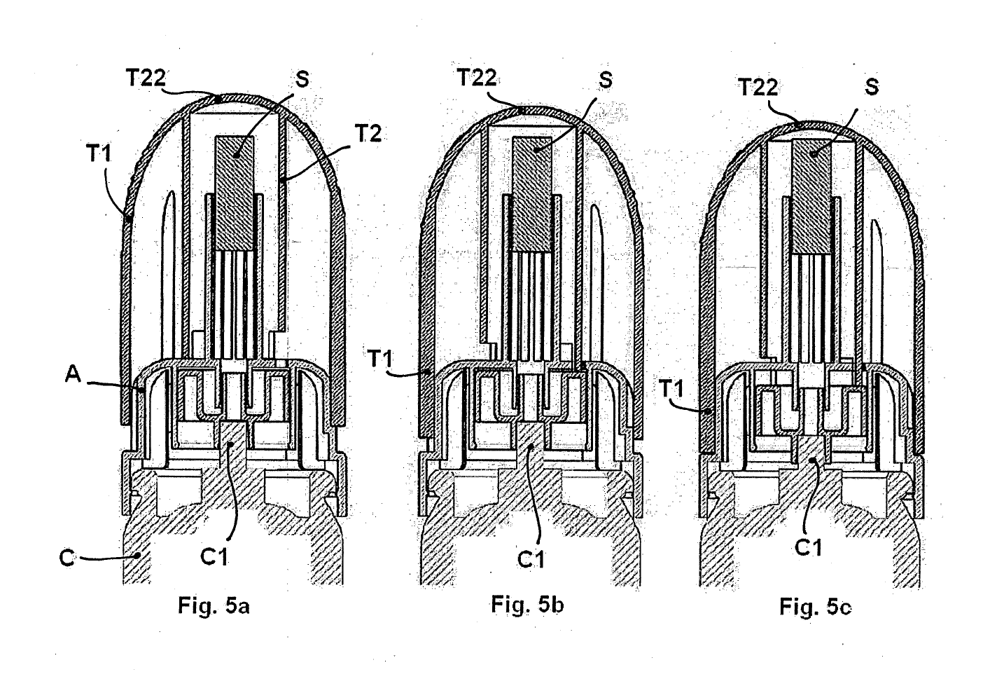

### DETAILED DESCRIPTION OF THE INVENTION

Referring to the figures, the new dispensing device can be integrally applied to pressurized containers (C) equipped with a manual valve (C1), of the sprayer type, and comprises a dispenser body (A) suited to be fixed to said container (C).

Said dispenser body (A) substantially comprises a convex or dome-shaped part (A2) suited to contain the upper end of the container and at least one duct or tube (A1) integral with and built in said cupola-shaped part (A2) for the outflow of the coolant contained in the container (C).

The inside of said tube (A1) is shaped in such a way as to comprise ribs (A1) suited to hold at least one cylindrical pad or filter (S) partially inserted in it.

In the preferred embodiment, in order to guarantee effective absorption and conveyance of the coolant, said cylindrical pad or filter (S) is made if polyurethane S518, with a diameter of approximately 7 mm and a length of 18 mm.

A valve-opening element (B) is interposed and fixed between said dispenser body (A) and said container (C), and comprises an annular portion (B1), which is inserted between partitions (A22) that are integral with the dispenser body (A) and which is at least one channel (B2) communicating with said valve (C1), at least partially inserted—coaxially—in said tube (A1) for application of the coolant.

Said valve-opening element (B) rests on said valve (C1) so that the downward travel of the valve-opening element (B) itself, for example caused by a downward force exerted on it, causes the opening of the valve (C1) and thus the outflow of the coolant that, through said channel (B2) provided inside said tube (A1), reaches said pad or filter (S) and is absorbed. The new dispensing device also includes at least one activator plug or cap (T) comprising a casing (T1) suited to be positioned to close the dispenser body (A), to which it can be removably connected, and an inner channel (T2), integral with the upper part of the cap (T), suited to house said tube (A1) of the dispenser body (A).

The cylindrical wall of said inner channel (T2) of the cap (T) comprises, on its lower edge, one or more teeth or projections (T21) suited to be inserted in corresponding through slots or holes (A21) made in said dome-shaped portion (A2) of said dispenser body (A), so that it can rest against a part (B3) of said valve-opening element (B), causing the valve (C1) to be opened and the fluid to be dispensed.

The outflow of fluid and the absorption of the same by the pad (5) makes the condensate deposit on the inner surface of the cap (T) in proximity of the top (T22) near the pad (S) itself, thus making it clear for the user that the pad is soaked.

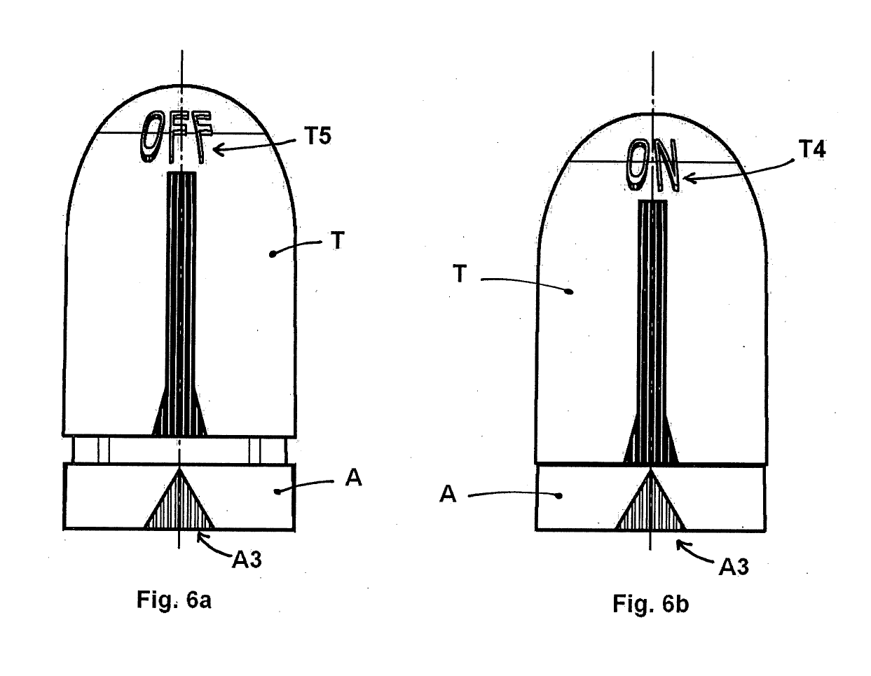

Furthermore, according to the invention, the inner surface of said casing (T1) of the cap (T) is provided with one or more ribs (T3), suited to be inserted in corresponding grooves present in said dispenser body (A), which serve as guides for the correct alignment and positioning of the cap (T) itself. In particular, according to the invention, said cap can be aligned and arranged on said dispenser body in two different positions. In position “OFF” (FIGS. 5a and 6a), the cap (T) inserted in said dispenser body is rotated so that said teeth (T21) are not aligned with said corresponding slots (A21) present in the dispenser body (A) and therefore they cannot be inserted in it and act on said valve-opening element (B).

This prevents the fluid from being dispensed.

In position “ON” (FIGS. 6b and 5b), the cap (T) is rotated by 180° with respect to position “OFF”, so that said teeth (T21) are aligned with said slots (A21).

If a force directed towards the container (C) is exerted on said cap (T), as shown in FIG. 5c, said teeth (T21) are inserted in the slots (A21) and, resting against said valve-opening element (B), cause the valve (CI) to be opened and the fluid to be dispensed.

To guide the user and allow him/her to correctly align the cap in position “OFF” or “ON”, said cap (T) and said dispenser body (A) are provided with reference marks that show how to align the cap (T) correctly and define the two “OFF” and “ON” positions.

In particular, said cap (T) is provided with an arrow and/or a reference mark to indicate position “ON” and with an arrow and/or reference mark (T4) to indicate position “OFF”, to be aligned with the reference mark (A3) provided on said dispenser body (A).

Therefore, with reference to the above description and the attached drawings, the following claims are expressed.

What is claimed is:

1. A dispensing device for a pressurized container of a coolant for cryotherapy, the container comprising at least one manual opening/closing valve, the dispensing device comprising:
at least one dispenser body suited to be permanently fixed to said container;
at least one valve-opening element positioned between said dispenser body and said container and resting on said valve;
at least one closing plug or cap suited to be removably positioned on and fixed to said dispenser body and comprising one or more teeth or projections resting on said valve-opening element such to cause the valve to open,
wherein at least one tube or duct for application of the coolant is permanently built in and integral with said dispenser body, and
wherein at least one applicator pad or filter is removably inserted at one end of the tube or duct.

2. The dispensing device according to claim 1, wherein said plug or cap comprises an external casing that surrounds and encloses said dispenser body and at least one internal channel, integral with the upper part of the casing and suitable for housing said tube, and wherein the upper part of said cap is in proximity of said pad or filter, the pad or filter being partially inserted in said tube, which is held in a correct position while the coolant is being dispensed.

3. The dispensing device according to claim 1, wherein said plug or cap is completely or partially made of a transparent plastic material that makes it possible to see from the outside a condensate forming when the coolant is dispensed.

4. The dispensing device according to claim 1, wherein said valve-opening element is shaped to define at least one channel communicating with an opening of said valve, the at least one channels being at least partially inserted in said tube and suited to convey said dispensed coolant, causing the dispensed coolant to reach said pad partially inserted in said tube.

5. The device according to claim 1, wherein said pad or filter is made of cellulose or open-cell foam or another material with a high capacity to absorb and convey the coolant.

6. The dispensing device according to claim 1, wherein said cap and/or said dispenser body and/or said container are provided with one or more ribs and/or grooves for a guided application with a correct alignment of said cap to said dispenser body.

7. The dispensing device according to claim 6, wherein said dispenser body shaped to define one or more through slots or holes for an insertion of one or more teeth or projections provided on a lower edge of an internal channel of said cap, and wherein one or more of said teeth or projections, inserted through said slots, rest on said valve-opening element to cause the valve to be opened and the coolant to be dispensed.

8. The dispensing device according to claim 7, wherein said ribs and/or grooves are disposed to allow said cap to be applied to said dispenser body and aligned in position “ON”, such that said teeth are aligned with said slots and are insertable in said slots by pressing the cap toward the container.

9. The dispensing device according to claim 7, wherein said ribs and/or grooves are configured to guide said cap and couple said cap to said dispenser body, aligning said cap in position “OFF”, rotated by a given angle with respect to said position “ON”, so that said teeth in position “OFF” are not aligned with said slots and thus are not insertable in said slots.

10. The dispensing device according to claim 1, wherein said cap and/or said dispenser body and/or said container are provided with one or more cuts, symbols, or reference marks for a correct alignment and arrangement of the cap in position “OFF” or “ON”.
